(19) United States
(12) Patent Application Publication
(10) Pub. No.: US 2006/0122191 A1
(43) Pub. Date: Jun. 8, 2006

(54) N-(indolethyl-)cycloamine compounds

(76) Inventors: Timo Heinrich, Gross-Umstadt (DE); Henning Bottcher, Darmstadt (DE); Kali Schiemann, seeheim-Jugenheim (DE); Gunter Holzemann, Seeheim-Jugenheim (DE); Christoph Van Amsterdam, Damstadt (DE); Gerd Bartoszyk, Weiterstadt (DE); Joachim Leibrock, Pfungstadt (DE); Christoph Seyfried, Seeheim-Jugenheim (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/539,516

(22) PCT Filed: Nov. 27, 2003
(86) PCT No.: PCT/EP03/13374

§ 371 Date: Jun. 17, 2005

(30) Foreign Application Priority Data
Dec. 17, 2002 (DE) 102 59 244.6

(51) Int. Cl. A61K 31/496; C07D 417/14; C07D 405/14

(52) U.S. Cl. 514/254.03; 514/254.09; 544/367; 544/373

(57) ABSTRACT

N-(indolethyl-)cycloamine compounds of the formula (I): in which R¹′, R¹″ X, Ar, and n have a meaning indicated in Claim 1, are serotonin reuptake inhibitors (SSRIs) and effectors of the serotonergic receptors 5-HT_{1A} and 5-HT_{2A}. They are therefore suitable for the prophylaxis or treatment of various diseases of the central nervous system, such as depression, dyskinesia, Parkinson's disease, dementia, strokes, schizophrenia, Alzheimer's disease, Lewy bodies dementia, Huntington's disease, Tourette's syndrome, anxiety, learning and memory impairment, pain, sleeping disorders and neurodegenerative diseases.

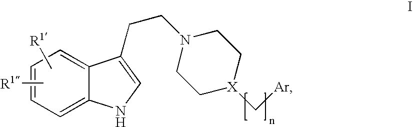

The invention relates to compounds of the formula I 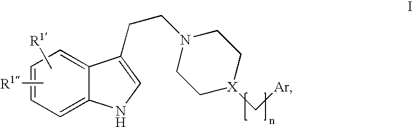
in which
R¹′, R¹″ each, independently of one another, denote H, CN, Hal, A, OA, OH, COR², CH₂R²,
R² denotes OH, OA, NH₂, NHA or NA₂,
R³ denotes H or A,
X denotes N or CH
A denotes unbranched or branched alkyl having 1-10 C atoms, in which one or two CH₂ groups may be replaced by O or S atoms and/or by —CH═CH— groups and/or also 1-7H atoms may be replaced by F,
Ar denotes unsaturated, partially or fully saturated, mono- or polycyclic homo- or heterocyclic system containing the hetero atoms O, N, S, which is unsubstituted or mono- or polysubstituted by Hal, A, OR³, N(R³)₂, NO₂, CN, COOR³, CON(R³)₂, NR³COA, NR³CON(R³)₂, NR³SO₂A, COR³, SO₂N(R³)₂, SO₂A,
Hal denotes F, Cl, Br or I and
n denotes 0, 1, 2, 3, 4,
   and pharmaceutically usable derivatives, solvates and stereoisomers thereof, including mixtures thereof in all ratios.

The invention was based on the object of finding novel compounds having valuable properties, in particular those which can be used for the preparation of medicaments.

It has been found that the compounds of the formula I and pharmaceutically usable derivatives, solvates and stereoisomers thereof, while being well tolerated, have valuable pharmacological properties since they have actions on the central nervous system. The compounds are, in particular, strong serotonin reuptake inhibitors (SSRIs). In addition, they are effectors of the serotonergic receptors 5-HT_{1A} and 5-HT_{2A}, where they exhibit a 5-HT_{1A}-agonistic action.

In-vitro evidence of interaction with the above-mentioned receptors can be provided, for example, as described in the following references:
5-HT_{1A}: Cossery J. M., Gozlan H., Spampinato U., Perdicakis C., Guillaumet G., Pichat L., Hamon M., 1987. The selective labeling of central 5-HT_{1A} receptor binding sites by [3H]5-methoxy-3-(di-n-propylamino)chroman. Eur. J. Pharmacol. 140,143-55.
5-HT_{2A}: Klockow M., Greiner H. E., Haase A., Schmitges C.-J., Seyfried C. 1986. Studies on the receptor profile of bisoprolol. Arzneimittelforschung 36,197-200.
SSRI: Wong, D T, Bymaster, F P, Mayle, D A. Reid, L R, Krushinski, J H, Robertson, D W. LY248686, a new inhibitor of serotonin and norepinephrine uptake. Neuropsychopharmacology 8, 23-33, 1993

The compounds of the formula I and physiologically acceptable salts thereof can be used for the prophylaxis or treatment of diseases of the central nervous system in which binding to serotonergic receptors, in particular 5-HT_{1A} and/or 5-HT_{2A} and/or inhibition of the reuptake of serotonin results in an improvement in the clinical picture.

Thus, the compounds of the formula I are suitable for the prophylaxis and treatment of various diseases of the central nervous system, such as, for example, depression, dyskinesia, Parkinson's disease, dementia, strokes or cerebral ischaemia, schizophrenia, Alzheimer's disease, Lewy bodies dementia, Huntington's disease, Tourette's syndrome, anxiety, learning and memory impairment, sleeping disorders, pain and neurodegenerative diseases.

In the treatment of the diseases described, the compounds according to the invention can also be employed in combination with other pharmacologically active compounds. The compounds according to the invention are administered at the same time as or before or after the other said substances.

Compounds of the formula I and salts and solvates thereof are also suitable as intermediates for the preparation of other medicament active ingredients.

The invention also relates to the stereoisomers (enantiomers and racemates thereof as well as diastereomers), hydrates and solvates of these compounds. Solvates of the compounds are taken to mean adductions of inert solvent molecules onto the compounds which form owing to their mutual attractive force. Solvates are, for example, mono- or dihydrates or alcoholates.

Pharmaceutically usable derivatives are taken to mean, for example, the salts of the compounds according to the invention, but also so-called prodrug compounds.

Prodrug derivatives are taken to mean compounds of the formula I which have been modified with, for example, alkyl or acyl groups, sugars or oligopeptides and which are rapidly cleaved in the organism to give the effective compounds according to the invention.

These also include biodegradable polymer derivatives of the compounds according to the invention, as described, for example, in Int. J. Pharm. 115, 61-67 (1995).

The invention also relates to mixtures of the compounds of the formula I according to the invention, for example mixtures of two diastereomers, for example in the ratio 1:1, 1:2, 1:3, 1:4, 1:5, 1:10, 1:100 or 1:1000. These are particularly preferably mixtures of stereoisomeric compounds. The invention relates to the compounds of the formula I and physiologically acceptable acid-addition salts thereof. The invention also relates to the solvates, for example hydrates or alcoholates, of these compounds.

The invention also relates to a process for the preparation of compounds of the formula I and pharmaceutically usable derivatives, salts and solvates thereof, characterised in that the following reaction steps are carried out:
a) For the preparation of the ethylindole starting material, an indole derivative of the formula VI 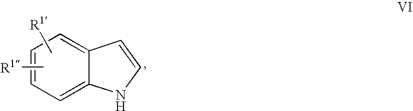
   in which R¹′ and R¹″ have a meaning indicated in Claim 1, is reacted with an acetyl halide which is substituted in the 2-position by a leaving group R which is suitable for nucleophilic substitution (such as, for example, Cl, Br, I, mesylate, tosylate, phenylsulfonate or trifluoroacetate) to give a compound of the formula V 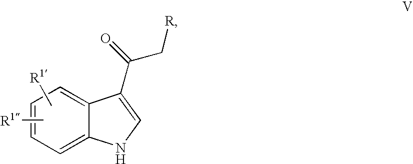
   which is then, after reduction to a compound of the formula IV 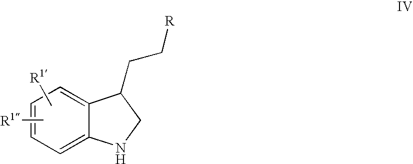
   oxidised further to give the ethylindole starting material of the formula III 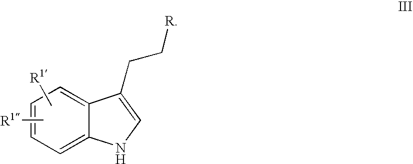
   (b) For the preparation of a compound of the formula I, the formylindole starting material of the formula III, in which R¹′ and R¹″ have a meaning indicated in Claim 1, and R is a leaving group which is suitable for nucleophilic substitutions, such as, for example, Cl, Br, I mesylate, tosylate, phenylsulfonate or trifluoroacetate, is brought to reaction with a cycloamine compound of the formula II 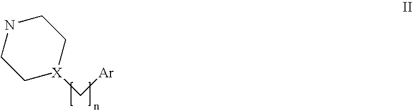
   in which X, Ar, and n have the meaning indicated in Claim, in the presence of a base.

A resultant base of the formula I can be converted into one of its salts by treatment with an acid.

The invention additionally relates to the ethylindole compound of the formula III as intermediate compounds for the preparation of the compounds of the formula I.

The invention also relates to the compounds of the formula I according to Claim 1 and pharmaceutically acceptable derivatives, salts or solvates thereof as medicaments.

The invention likewise relates to the compounds of the formula I according to Claim 1 and pharmaceutically acceptable derivatives, salts or solvates thereof as serotonin reuptake inhibitors and effectors of the serotonergic receptors 5-HT_{1A} and 5-HT_{2A}.

The invention likewise relates to the compounds of the formula I according to Claim 1 and pharmaceutically acceptable derivatives, salts or solvates thereof as serotonin reuptake inhibitors and effectors of the serotonergic receptors 5-HT_{1A} and 5-HT_{2A} for the prophylaxis or treatment of various diseases of the central nervous system, such as depression, dyskinesia, Parkinson's disease, dementia, strokes, schizophrenia, Alzheimer's disease, Lewy bodies dementia, Huntington's disease, Tourette's syndrome, anxiety, learning and memory impairment, sleeping disorders, pain and neurodegenerative diseases.

The invention furthermore relates to the use of compounds of the formula I for the preparation of medicaments, in particular medicaments which are employed for the treatment of diseases based on a dysfunction of serotonin reuptake and/or serotonergic receptors, such as the receptors 5-HT_{1A} and/or 5-HT_{2A}.

The invention likewise relates to the use of compounds of the formula I according to Claim 1 and/or physiologically acceptable salts or solvates thereof for the preparation of a medicament, in particular for the preparation of a medicament for the prophylaxis or treatment of diseases in which inhibition of serotonin reuptake and/or binding of one or more active ingredients present in the said medicament to serotonergic receptors, such as the receptor 5-HT_{1A} and/or 5-HT_{2A}, results in an improvement in the clinical picture.

The invention furthermore relates to the use of compounds of the formula I according to Claim 1 and/or of physiologically acceptable salts and solvates thereof for the preparation of a medicament for the prophylaxis or treatment of various diseases of the central nervous system, such as depression, dyskinesia, Parkinson's disease, dementia, strokes, schizophrenia, Alzheimer's disease, Lewy bodies dementia, Huntington's disease, Tourette's syndrome, anxiety, learning and memory impairment, pain, sleeping disorders and neurodegenerative diseases.

Finally, the invention relates to pharmaceutical compositions comprising the compounds of the formula I and pharmaceutically acceptable derivatives, salts or solvates thereof, and to a process for the preparation of the pharmaceutical compositions.

The compounds of the formula I may have one or more chiral centres and may therefore occur in various stereoisomeric forms. The formula I encompasses all these forms.

For all radicals which can occur more than once, such as A, R² or R³, their meanings are independent of one another.

A denotes alkyl, is unbranched (linear) or branched, and has 1, 2, 3, 4, 5, 6, 7, 8, 9 or 10 C atoms.

A preferably denotes methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, furthermore preferably, for example, trifluoromethyl.

A very particularly preferably denotes alkyl having 1-6 C atoms, preferably methyl, ethyl, propyl, isopropyl, butyl, isobutyl, sec-butyl, tert-butyl, pentyl, hexyl, trifluoromethyl, pentafluoroethyl or 1,1,1-trifluoroethyl.

A furthermore denotes cycloalkyl, preferably cyclopropyl, cyclobutyl, cyclopentyl, cyclohexyl, cycloheptyl, cyclooctyl or 2,6,6-trimethylbicyclo-3.1.1-heptyl, but likewise mono- or bicyclic terpenes, preferably p-menthane, menthol, pinane, bornane or camphor, where every known stereoisomeric form is included, or adamantyl. For camphor, this denotes both L-camphor and D-camphor.

Ar denotes an unsaturated, partially or fully saturated, mono- or polycyclic homo- or heterocyclic system containing the hetero atoms O, N, S, which is unsubstituted or mono- or polysubstituted by Hal, A, OR³, N(R³)₂, NO2, CN, COOR³, CON(R³)₂, NR³COA, NR³CON(R³)₂, NR³SO₂A, COR³, SO2N(R³)2, SO₂A.

Particularly preferred homocyclic systems are unsubstituted or substituted phenyl, naphthyl or biphenyl, specifically preferably phenyl, o-, m- or p-tolyl, o-, m- or p-ethylphenyl, o-, m- or p-propylphenyl, o-, m- or p-isopropylphenyl, o-, m- or p-tert-butylphenyl, o-, m- or p-trifluoromethylphenyl, o-, m- or p-aminophenyl, o-, m- or p-hydroxyphenyl, o-, m- or p-nitrophenyl, o-, m- or p-(trifluoromethoxy)phenyl, o-, m- or p-cyanophenyl, o-, m- or p-methoxyphenyl, o-, m- or p-ethoxyphenyl, o-, m- or p-fluorophenyl, o-, m- or p-bromophenyl, o-, m- or p-chlorophenyl, o-, m- or p-(difluoromethoxy)phenyl, o-, m- or p-(fluoromethoxy)phenyl, furthermore preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2-chloro-3-methyl-, 2-chloro-4-methyl-, 2-chloro-5-methyl-, 2-chloro-6-methyl-, 2-methyl-3-chloro-, 2-methyl-4-chloro-, 2-methyl-5-chloro-, 2-methyl-6-chloro-, 3-chloro-4-methyl-, 3-chloro-5-methyl- or 3-methyl-4-chlorophenyl, 2-bromo-3-methyl-, 2-bromo-4-methyl-, 2-bromo-5-methyl-, 2-bromo-6-methyl-, 2-methyl-3-bromo-, 2-methyl-4-bromo-, 2-methyl-5-bromo-, 2-methyl-6-bromo-, 3-bromo-4-methyl-, 3-bromo-5-methyl- or 3-methyl-4-bromophenyl, 2,4- or 2,5-dinitrophenyl, 2,5- or 3,4-dimethoxyphenyl, 3-nitro-4-chlorophenyl, 2,3,4-, 2,3,5-, 2,3,6-, 2,4,6- or 3,4,5-trichlorophenyl, 2,4,6-tri-tert-butylphenyl, furthermore preferably 2-nitro-4-(trifluoromethyl)-phenyl, 3,5-di-(trifluoromethyl)phenyl, 2,5-dimethylphenyl, 2-hydroxy-3,5-dichlorophenyl, 2-fluoro-5- or 4-fluoro-3-(trifluoromethyl)phenyl, 4-chloro-2- or 4-chloro-3-(trifluoromethyl)-, 2-chloro-4- or 2-chloro-5-(trifluoromethyl)-phenyl, 4-bromo-2- or 4-bromo-3-(trifluoromethyl)phenyl, p-iodophenyl, 2-nitro-4-methoxyphenyl, 2,5-dimethoxy-4-nitrophenyl, 2-methyl-5-nitrophenyl, 2,4-dimethyl-3-nitrophenyl, 4-fluoro-3-chlorophenyl, 4-fluoro-3,5-dimethylphenyl, 2-fluoro-4-bromophenyl, 2,5-difluoro-4-bromophenyl, 2,4-dichloro-5-methylphenyl, 3-bromo-6-methoxyphenyl, 3-chloro-6-methoxyphenyl, 2-methoxy-5-methylphenyl or 2,4,6-triisopropylphenyl, 2-, 3 or 4-methoxycarbonylphenyl, 2-, 3 or 4-ethoxycarbonylphenyl, 2-, 3 or 4-propoxycarbonylphenyl, 2-, 3 or 4-butoxycarbonylphenyl, 2-, 3 or 4-pentoxycarbonylphenyl, 2-, 3 or 4-hexoxycarbonylphenyl, 2-, 3 or 4-methylaminocarbonylphenyl, 2-, 3 or 4-ethylaminocarbonylphenyl, 2-, 3 or 4-propylaminocarbonylphenyl, 2-, 3 or 4-butylaminocarbonylphenyl, 2-, 3 or 4-pentylaminocarbonylphenyl, 2-, 3 or 4-hexylaminocarbonylphenyl, 2,3-, 2,4- or 2,5-dimethylaminocarbonylphenyl or 2,3-, 2,4- or 2,5-diethylaminocarbonylphenyl.

Particularly preferred heterocyclic systems are unsubstituted or substituted indole, benzofuran, benzodioxolane, benzodioxin or benzothiadiazole.

Hal denotes fluorine, chlorine, bromine or iodine, particularly preferably fluorine, chlorine or bromine.

R¹′, R¹″ each, independently of one another, denotes H, CN, Hal, A, OA, OH, COR², CH₂R², where A, Hal and R² have one of the meanings described. R¹′, R¹″ are, in particular, hydrogen, hydroxyl, methoxy, ethoxy, propoxy, butoxy, pentyloxy, hexyloxy, trifluoromethoxy, fluorine, chlorine, bromine, iodine, cyano, methoxycarbonyl, ethoxycarbonyl, propoxycarbonyl, butoxycarbonyl, pentoxycarbonyl, hexoxycarbonyl, methylaminocarbonyl, ethylaminocarbonyl, propylaminocarbonyl, butylaminocarbonyl, pentylaminocarbonyl or hexylaminocarbonyl. Particularly preferably, R¹′ is cyano and R¹″ is simultaneously hydrogen.

R² denotes OH, OA, NH₂, NHA or NA₂, where A has the above-mentioned meaning.

R³ denotes hydrogen or A, where A has one of the above-mentioned meanings. R³ is preferably hydrogen, methyl, ethyl, n-propyl, i-propyl, n-butyl, i-butyl or t-butyl. R³ is particularly preferably hydrogen.

n is 0, 1, 2, 3, 4. n is preferably 0, 1 or 2. n is particularly preferably =2.

In particular, the invention relates to the compounds of the formula I in which at least one of the said radicals has one of the preferred meanings indicated above. For a given compound of the formula I, the following principle applies: the more of the radicals present therein have a preferred meaning, the more the compound is preferred overall. Some preferred groups of compounds can be expressed by the following sub-formulae Ia to If, which conform to the formula I and in which the radicals not designated in greater detail have the meaning indicated for the formula I, but in which

| | | |
|---|---|---|
| in Ia | R^{1′} | denotes cyano, |
| | R^{1″} | denotes hydrogen, |
| | X | denotes N, |
| | n | denotes 0, 1 or 2; |
| in Ib | R^{1′} | denotes cyano, |
| | R^{1″} | denotes hydrogen, |
| | X | denotes N, |
| | n | denotes 0, 1 or 2, |
| | Ar | denotes phenyl which is unsubstituted or substituted |
| | | as indicated in Claim 1; |
| in Ic | R^{1′} | denotes cyano, |
| | R^{1″} | denotes hydrogen, |
| | X | denotes N, |
| | n | denotes 0, 1 or 2, |
| | Ar | denotes naphthyl which is unsubstituted or substituted |
| | | as indicated in Claim 1; |
| in Id | R1′ | denotes cyano, |
| | R1″ | denotes hydrogen, |
| | X | denotes N, |
| | n | denotes 0, 1 or 2, |
| | Ar | denotes indolyl, benzofuryl or benzodioxolyl, each |
| | | of which is unsubstituted or substituted as indicated |
| | | in Claim 1; |
| in Ie | R1′ | denotes cyano, |
| | R1″ | denotes hydrogen, |
| | X | denotes N, |
| | n | denotes 0, 1 or 2, |
| | Ar | denotes benzodioxinyl which is unsubstituted or |
| | | substituted as indicated in Claim 1; |
| in If | R1′ | denotes cyano, |
| | R1″ | denotes hydrogen, |
| | X | denotes N, |
| | n | denotes 0, 1 or 2, |
| | Ar | denotes benzothiadiazolyl which is unsubstituted or |
| | | substituted as indicated in Claim 1; |

In particular, the invention relates to the following compounds of the formula I:
a) 3-{2-[4-(2,3-dihydrobenzo-1,4-dioxin-5-yl)piperazin-1-yl]ethyl}-1H-indole-5-carbonitrile and
b) 3-[2-(4-benzo-1,2,5-thiadiazol-4-ylpiperazin-1-yl)ethyl]-1H-indole-5-carbonitrile
   and pharmaceutically usable derivatives, solvates and stereoisomers thereof, including mixtures thereof in all ratios.

The compounds of the formula I and also the starting materials for the preparation thereof are prepared by methods known per se, as described in the literature (for example in standard works, such as Houben-Weyl, Methoden der Organischen Chemie [Methods of Organic Chemistry], Georg Thieme Verlag, Stuttgart; Organic Reactions, John Wiley & Sons, Inc., New York), to be precise under reaction conditions as are known and suitable for the said reactions. Use can also be made here of variants known per se which are not explained in greater detail here.

The starting materials for the claimed process can also be formed in situ by not isolating them from the reaction mixture, but instead immediately converting them further into the compounds of the formula I. On the other hand, it is possible to carry out the reaction stepwise.

The N-(indolethyl-)cycloamine compounds of the formula I can preferably be obtained by reacting a formylindole starting material of the formula III with a cycloamine compound of the formula II as follows:

A compound of the formula II is dissolved in an inert solvent together with a compound of the formula III and an organic base and subsequently stirred at elevated temperature. The reaction mixture is subsequently poured onto ice. The crystals forming in the process are filtered off with suction, washed and optionally recrystallised.

The formylindole starting materials of the formula III and the cycloamine compounds of the formula II are generally known and commercially available; the compounds of the formulae II and III that are not known can easily be prepared analogously to known compounds. The preparation of the compound of the formula III 3-(2-chloroeth-1-yl)-1H-indole-5-carbonitrile and the compound of the formula 114-piperazin-1-ylbenzothiadiazole are described in Examples 1 and 2. The compound of the formula II 2,3-dihydrobenzo-1,4-dioxin-5-yl)piperazine is commercially available.

The reaction described above is generally carried out in an inert solvent, in the presence of an acid-binding agent, preferably an organic base, such as triethylamine, dimethylaniline, pyridine or quinoline, an alkali or alkaline-earth metal hydroxide, carbonate or bicarbonate or another salt of a weak acid of the alkali or alkaline-earth metals, preferably of potassium, sodium, calcium or caesium.

Examples of suitable inert solvents for the above-described reactions are hydrocarbons, such as hexane, petroleum ether, benzene, toluene or xylene; chlorinated hydrocarbons, such as trichloroethylene, 1,2-dichloroethane, carbon tetrachloride, chloroform or dichloromethane; ethers, such as diethyl ether, diisopropyl ether, tetrahydrofuran (THF) or dioxane; glycol ethers, such as ethylene glycol monomethyl or monoethyl ether, ethylene glycol dimethyl ether (diglyme); ketones, such as acetone or butanone; amides, such as acetamide, N-methylpyrrolidone (NMP), dimethylacetamide or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

Depending on the conditions used, the reaction temperature for the above-described reactions is between about −10 and 200°, normally between 60° and 180°, preferably between 100° and 140°, particularly preferably 120°. Depending on the conditions used, the reaction time is between a few minutes and several days.

A resultant base of the formula I can be converted into the associated acid-addition salt using an acid. Suitable acids for this reaction are those which give physiologically acceptable salts. Thus, it is possible to use inorganic acids, for example sulfuric acid, hydrohalic acids, such as hydrochloric acid or hydrobromic acid, phosphoric acids, such as orthophosphoric acid, nitric acid, sulfamic acid, furthermore organic acids, specifically aliphatic, alicyclic, araliphatic, aromatic or heterocyclic mono- or polybasic carboxylic, sulfonic or sulfuric acids, such as formic acid, acetic acid, propionic acid, pivalic acid, diethylacetic acid, malonic acid, succinic acid, pimelic acid, fumaric acid, maleic acid, lactic acid, tartaric acid, malic acid, benzoic acid, salicylic acid, 2-phenylpropionic acid, citric acid, gluconic acid, ascorbic acid, nicotinic acid, isonicotinic acid, methane- or ethanesulfonic acid, ethanedisulfonic acid, 2-hydroxyethanesulfonic acid; benzenesulfonic acid, p-toluenesulfonic acid, naphthalenemono- and -disulfonic acids, laurylsulfuric acid.

The free bases of the formula I can, if desired, be liberated from their salts by treatment with strong bases, such as sodium hydroxide, potassium hydroxide, sodium carbonate or potassium carbonate, so long as no further acidic groups are present in the molecule.

Compounds of the formula I can furthermore be obtained by liberating compounds of the formula I from one of their functional derivatives by treatment with a solvolysing or hydrogenolysing agent.

Preferred starting materials for the solvolysis or hydrogenolysis are those which conform to the formula I, but contain corresponding protected amino and/or hydroxyl groups instead of one or more free amino and/or hydroxyl groups, preferably those which carry an amino-protecting group instead of an H atom bonded to an N atom, in particular those which carry an R′-N group, in which R′ denotes an amino-protecting group, instead of an HN group, and/or those which carry a hydroxyl-protecting group instead of the H atom of a hydroxyl group, for example those which conform to the formula I, but carry a —COOR″ group, in which R″ denotes a hydroxyl-protecting group, instead of a —COOH group.

Preferred starting materials are also the oxadiazole derivatives, which can be converted into the corresponding amidino compounds.

It is also possible for a plurality of—identical or different—protected amino and/or hydroxyl groups to be present in the molecule of the starting material. If the protecting groups present are different from one another, they can in many cases be cleaved off selectively.

The term “amino-protecting group” is known in general terms and relates to groups which are suitable for protecting (blocking) an amino group against chemical reactions, but which are easy to remove after the desired chemical reaction has been carried out elsewhere in the molecule. Typical of such groups are, in particular, unsubstituted or substituted acyl, aryl, aralkoxymethyl or aralkyl groups. Since the amino-protecting groups are removed after the desired reaction (or reaction sequence), their type and size is furthermore not crucial; however, preference is given to those having 1-20, in particular 1-8, C atoms. The term “acyl group” is to be understood in the broadest sense in connection with the present process. It includes acyl groups derived from aliphatic, araliphatic, aromatic or heterocyclic carboxylic acids or sulfonic acids, and, in particular, alkoxycarbonyl, aryloxycarbonyl and especially aralkoxycarbonyl groups. Examples of such acyl groups are alkanoyl, such as acetyl, propionyl, butyryl; aralkanoyl, such as phenylacetyl; aroyl, such as benzoyl, tolyl; aryloxyalkanoyl, such as POA; alkoxycarbonyl, such as methoxycarbonyl, ethoxycarbonyl, 2,2,2-trichloroethoxycarbonyl, BOC (tert-butoxycarbonyl), 2-iodoethoxycarbonyl; aralkoxycarbonyl, such as CBZ (“carbobenzoxy”), 4-methoxybenzyloxycarbonyl, FMOC; arylsulfonyl, such as Mtr. Preferred amino-protecting groups are BOC and Mtr, furthermore CBZ, Fmoc, benzyl and acetyl.

Furthermore, free amino groups can be acylated in a conventional manner using an acid chloride or anhydride or alkylated using an unsubstituted or substituted alkyl halide, or reacted with CH₃—C(═NH)—OEt, advantageously in an inert solvent, such as dichloromethane or THF, and/or in the presence of a base, such as triethylamine or pyridine, at temperatures between −60 and +30°.

The term “hydroxyl-protecting group” is likewise known in general terms and relates to groups which are suitable for protecting a hydroxyl group against chemical reactions, but which are easy to remove after the desired chemical reaction has been carried out elsewhere in the molecule. Typical of such groups are the above-mentioned unsubstituted or substituted aryl, aralkyl or acyl groups, furthermore also alkyl groups. The nature and size of the hydroxyl-protecting groups is not crucial since they are removed again after the desired chemical reaction or reaction sequence; preference is given to groups having 1-20, in particular 1-10, C atoms. Examples of hydroxyl-protecting groups are, inter alia, benzyl, 4-methoxybenzyl, p-nitrobenzoyl, p-toluenesulfonyl, tert-butyl and acetyl, where benzyl and tert-butyl are particularly preferred.

The compounds of the formula I are liberated from their functional derivatives—depending on the protecting group used—for example using strong acids, advantageously using TFA or perchloric acid, but also using other strong inorganic acids, such as hydrochloric acid or sulfuric acid, strong organic carboxylic acids, such as trichloroacetic acid, or sulfonic acids, such as benzene- or p-toluenesulfonic acid. The presence of an additional inert solvent is possible, but is not always necessary. Suitable inert solvents are preferably organic, for example carboxylic acids, such as acetic acid, ethers, such as tetrahydrofuran or dioxane, amides, such as DMF, halogenated hydrocarbons, such as dichloromethane, furthermore also alcohols, such as methanol, ethanol or isopropanol, and water. Mixtures of the above-mentioned solvents are furthermore suitable. TFA is preferably used in excess without addition of a further solvent, perchloric acid is preferably used in the form of a mixture of acetic acid and 70% perchloric acid in the ratio 9:1. The reaction temperatures for the cleavage are advantageously between about 0 and about 50°, preferably between 15 and 30° (room temperature, RT).

The BOC, OBut and Mtr groups can, for example, preferably be cleaved off using TFA in dichloromethane or using approximately 3 to 5N HCl in dioxane at 15-30°, the FMOC group using an approximately 5 to 50% solution of dimethylamine, diethylamine or piperidine in DMF at 15-30°.

Hydrogenolytically removable protecting groups (for example CBZ, benzyl or the liberation of the amidino group from its oxadiazole derivative)) can be cleaved off, for example by treatment with hydrogen in the presence of a catalyst (for example a noble-metal catalyst, such as palladium, advantageously on a support, such as carbon). Suitable solvents here are those indicated above, in particular, for example, alcohols, such as methanol or ethanol, or amides, such as DMF. The hydrogenolysis is generally carried out at temperatures between about 0 and 100° and pressures between about 1 and 200 bar, preferably at 20-30° and 1-10 bar. Hydrogenolysis of the CBZ group succeeds well, for example, on 5 to 10% Pd/C in methanol or using ammonium formate (instead of hydrogen) on Pd/C in methanol/DMF at 20-30°.

Esters can be saponified, for example, using acetic acid or using NaOH or KOH in water, water/THF or water/dioxane, at temperatures between 0 and 100°.

Further methods for the removal of protecting groups is described, for example, in Theodora W. Green, Peter G. M. Wuts: Protective Groups in Organic Synthesis, 3rd Edition John Wiley & Sons (1999).

Compounds of the formula I according to the invention may be chiral owing to their molecular structure and may accordingly occur in various enantiomeric forms. They can therefore exist in racemic or in optically active form. Since the pharmaceutical activity of the racemates or stereoisomers of the compounds according to the invention may differ, it may be desirable to use the enantiomers. In these cases, the end product or even the intermediates can be separated into enantiomeric compounds by chemical, biochemical or physical measures known to the person skilled in the art or even employed as such in the synthesis.

In the case of racemic amines, diastereomers are formed from the mixture by reaction with an optically active resolving agent. Examples of suitable resolving agents are optically active acids, such as the R and S forms of tartaric acid, diacetyltartaric acid, dibenzoyltartaric acid, mandelic acid, malic acid, lactic acid, suitably N-protected amino acids (for example N-benzoylproline or N-benzenesulfonylproline), or the various optically active camphorsulfonic acids. Also advantageous is chromatographic enantiomer resolution with the aid of an optically active resolving agent (for example dinitrobenzoylphenylglycine, cellulose triacetate or other derivatives of carbohydrates or chirally derivatised methacrylate polymers immobilised on silica gel). Suitable eluents for this purpose are aqueous or alcoholic solvent mixtures, such as, for example, hexane/isopropanol/acetonitrile, for example in the ratio 82:15:3.

An elegant method for the resolution of racemates containing ester groups (for example acetyl esters) is the use of enzymes, in particular esterases.

The invention furthermore relates to the use of the compounds of the formula I and/or physiologically acceptable salts thereof for the preparation of a medicament (pharmaceutical composition), in particular by non-chemical methods. They can be brought into a suitable dosage form here together with at least one solid, liquid and/or semi-liquid excipient or adjuvant and, if desired, in combination with one or more further active ingredients.

These compositions can be used as medicaments in human or veterinary medicine. Suitable excipients are organic or inorganic substances which are suitable for enteral (for example oral), parenteral or topical administration and do not react with the novel compounds, for example water, vegetable oils, benzyl alcohols, alkylene glycols, polyethylene glycols, glycerol triacetate, gelatine, carbohydrates, such as lactose or starch, magnesium stearate, talc, Vaseline. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, suitable for topical application are ointments, creams or powders. The novel compounds may also be lyophilised and the resultant lyophilisates used, for example, for the preparation of injection preparations. The compositions indicated may be sterilised and/or comprise adjuvants, such as lubricants, preservatives, stabilisers and/or wetting agents, emulsifying agents, salts for modifying the osmotic pressure, buffer substances, colorants, flavours and/or a plurality of further active ingredients, for example one or more vitamins.

In general, the substances according to the invention are administered analogously to known, commercially available preparations, preferably in doses between about 100 μg and 100 mg, in particular between 1 and 40 mg, per dosage unit. The daily dose is preferably between about 1 μg and 1 mg per kg of body weight.

The specific dose for each individual patient depends on a very wide variety of factors, for example on the efficacy of the specific compound employed, on the age, body weight, general state of health, sex, on the diet, on the time and method of administration, on the excretion rate, medicament combination and severity of the particular disease to which the therapy applies.

Oral administration is preferred.

The invention thus also relates to medicaments comprising at least one compound of the formula I and/or pharmaceutically usable derivatives, solvates and stereoisomers thereof, including mixtures thereof in all ratios.

The invention furthermore relates to medicaments comprising at least one compound of the formula I and/or pharmaceutically usable derivatives, solvates and stereoisomers thereof, including mixtures thereof in all ratios, and at least one further medicament active ingredient.

The invention also relates to a set (kit) consisting of separate packs of
(a) an effective amount of a compound of the formula I and/or pharmaceutically usable derivatives, solvates and stereoisomers thereof, including mixtures thereof in all ratios, and
(b) an effective amount of a further medicament active ingredient.

The set comprises suitable containers, such as boxes, individual bottles, bags or ampoules. The set may, for example, comprise separate ampoules each containing an effective amount of a compound of the formula I and/or pharmaceutically usable derivatives, solvates and stereoisomers thereof, including mixtures thereof in all ratios,

and an effective amount of a further medicament active ingredient in dissolved or lyophilised form.

The invention furthermore relates to the use of compounds of the formula I and/or pharmaceutically usable derivatives, solvates and stereoisomers thereof, including mixtures thereof in all ratios,

for the preparation of a medicament for the prophylaxis or treatment of various diseases of the central nervous system, such as depression, dyskinesia, Parkinson's disease, dementia, strokes, schizophrenia, Alzheimer's disease, Lewy bodies dementia, Huntington's disease, Tourette's syndrome, anxiety, learning and memory impairment, pain, sleeping disorders and neurodegenerative diseases, in combination with at least one further medicament active ingredient.

Even without further comments, it is assumed that a person skilled in the art will be able to utilise the above description in the broadest scope. The preferred embodiments should therefore merely be regarded as descriptive disclosure which is absolutely not limiting in any way.

The characterisation of the resultant substances can be carried out by, for example, by ESI-MS (electrospray ionisation mass spectrometry (M+H)⁺), elemental analysis, TLC (thin-layer chromatography) and melting-point determination. Above and below, all temperatures are indicated in ° C. The values of the elementals are calculated on hydrochloride, unless indicated otherwise.

### EXAMPLE 1

### Synthesis of the Ethylindole Starting Material 3-(2-chloroeth-1-yl)-1H-indole-5-carbonitrile

a) With nitrogen aeration, 50 g (0.35 mol) of 7-cyanoindole are initially introduced in 500 ml of 1,2-dichloromethane, 47.7 g (0.42 mol) of 2-chloroacetyl chloride in 500 ml of 1,2-dichloroethane are added, and the batch is cooled to −15° C. At the indicated temperature, 56.3 g (0.42 mol) of aluminium trichloride are added, and the mixture is stirred for a further 2 h before the batch is warmed to RT. The batch is subsequently poured onto ice with stirring, and the precipitated crystals are filtered off with suction. After washing with water, drying is carried out for 12 h at 100° C. under reduced pressure. 60 g of the resultant crystals are recrystallised from 300 ml of DMF, giving about 20 g of beige-coloured crystals which exhibit an Rf value of 0.4 in the TLC in ethyl acetate.

[M+H]+ 219 (ESI-MS)

b) 2 g (9 mmol) of the acylated indole from Example 1 (a) are stirred at RT for 96 h together with 2.7 g (23 mmol) of triethylsilane in 20 ml of trifluoroacetic acid. The reaction mixture is poured into ice-water and adjusted to pH 10 using conc. NaOH. The resultant crystalline starting material is filtered off with suction, and the mother liquor is extracted to exhaustion with ethyl acetate. The organic phase is acidified using concentrated hydrochloric acid and extracted with water. The organic phase is discarded, and the aqueous phase is rendered alkaline again using conc. NaOH and extracted with ethyl acetate. After drying over sodium sulfate and evaporation of the organic phase, the residue is purified by chromatography using ethyl acetate over a silica gel column. The resultant pale oil (about 18 g) exhibits an Rf value of 0.6 in ethyl acetate.

[M+H]+ 207 (ESI-MS).

c) 500 mg (2.4 mmol) of the oil obtained in accordance with Example 1 (b) are dissolved in 300 ml of CH₂Cl₂, and 2.1 g (24 mmol) of MnO₂ are added to the solution. The reaction mixture is stirred at RT (room temperature) for 12 h, filtered off with suction through kieselguhr and evaporated. The residue becomes solid in the process. The resultant approx. 400 mg of crystalline 3-(2-chloroeth-1-yl)-1H-indole-5-carbonitrile exhibit an RF value of 0.1 in the toluene/methanol/triethylamine=7:2:1 thin-layer system.

[M+H]+ 205 (ESI-MS)

### EXAMPLE 2

### Synthesis of the Piperazine Starting Material 4-piperazin-1-yl-benzothiadiazole

a) Commercially available 4-nitrobenzothiadiazole (105 g, 0.58 mol) is dissolved in 2 l of ethanol, and 400 ml of glacial acetic acid are added. The solution is warmed to 50° C. At this temperature, 110 g (0.3 mol) of iron turnings are introduced in portions over the course of one hour. When the addition is complete, the batch is heated under reflux for six hours. When the TLC shows complete conversion, the mixture is cooled and filtered, and the filtrate is concentrated and partitioned between 3 l of water and 3 l of tert-butyl methyl ether. After extraction to exhaustion, the organic phase is washed with sodium hydrogencarbonate solution and dried over sodium sulfate and activated carbon. The residue subsequently obtained (55 g) is chromatographed over 1 kg of silica gel using dichloromethane, giving about 50 g of 4-aminobenzothiadiazole having a melting point of 67° C.

b) 3 g (19.8 mmol) of the amine prepared in accordance with Example 2(a) and 5.5 g (30.2 mmol) of bis(2-chloroethyl)ammonium chloride and 4.5 ml (26.5 mmol) of N-ethyldiisopropylamine are dissolved in 25 ml of chlorobenzene and heated at 150° C. for 30 h. After the solvent has been distilled off, the residue is stirred with 50 ml of methanol, filtered, and the residue is evaporated. 1.5 g of the desired piperazine having a melting range of 242-245° C. crystallise from acetone.

### EXAMPLE 3

### Synthesis of 3-{2-[4-(2,3-dihydrobenzo-1,4-dioxin-5-yl)-piperazin-1-yl]ethyl}-1H-indole-5-carbonitrile

1 g (5 mmol) of 3-(2-chloroeth-1-yl)-1H-indole-5-carbonitrile obtained in accordance with Example 1, 1.3 g (5 mmol) of commercially available 2,3-dihydrobenzo-1,4-dioxin-5-yl)piperazine and 1.9 g (15 mmol) of ethyldiisopropylamine are stirred at 120° C. for 12 h in 50 ml of N-methylpyrrolidinone. For work-up, the reaction mixture is introduced dropwise into ice-water adjusted to pH=10 using sodium hydroxide solution, during which beige-coloured crystals deposit. The mixture is stirred at RT for a further 1 h, the crystals are filtered off with suction and left to dry in air for 10 h. The crystals are subsequently dissolved in ethyl acetate, washed with water, dried using sodium sulfate and evaporated after the salt has been filtered off. The residue is chromatographed over a silica gel column using ethyl acetate/methanol 9:1. The product fractions are evaporated, and the resultant residue is dissolved in acetone. Hydrochloric acid (c=1 mol/l) is added dropwise to this solution until a pH of 3 is achieved. The resultant yellow crystals are filtered off with suction, washed with acetone and dried in air, giving about 0.5 g of brown crystals, which have an Rf value of 0.5 in an ethyl acetate/methanol=8:2 thin-layer chromatography system and a melting point of 277.5-278.5° C.

[M+H]+ 389 (ESI-MS)

| Elemental | | | | |
|---|---|---|---|---|
| analysis: | C | H | Cl | N |
| Sought: | 65.01 | 5.93 | 8.34 | 13.18 |
| Found: | 63.8 | 5.8 | 8.8 | 12.8 |

### EXAMPLE 4

### Synthesis of 3-[2-(4-benzo-1,2,5-thiadiazol-4-ylpiperazin-1-yl)-ethyl]-1H-indole-5-carbonitrile

300 mg (1.5 mmol) of 3-(2-chloroeth-1-yl)-1H-indole-5-carbonitrile obtained in accordance with Example 1 and 300 mg (1.6 mmol) of 4-piperazin-1-yl-benzothiadiazole obtained in accordance with Example 2 are stirred at 120° C. for 36 h in 200 ml of N-methylpyrrolidinone. After work-up as described in Example 3, about 15 mg of yellow crystals having an Rf value of 0.5 in ethyl acetate/methanol=8:2 are obtained.

[M+H]⁺ 389 (ESI-MS)

| Elemental | | | | | |
|---|---|---|---|---|---|
| analysis: | C | H | Cl | N | S |
| Sought: | 59.35 | 4.98 | 8.34 | 19.78 | 7.55 |
| Found: | 57.8 | 5.1 | — | 18.8 | 6.2 |

### EXAMPLE 5

### Synthesis of Further Compounds of the Formula I

The following compounds of the formula I according to the invention are obtained analogously to Examples 3 and 4 from the reaction of 3-(2-chloroeth-1-yl)-1H-indole-5-carbonitrile and a corresponding piperazine derivative of the formula II:

| | [M +H]⁺ |
|---|---|
| Compound | (ESI-MS) |
| 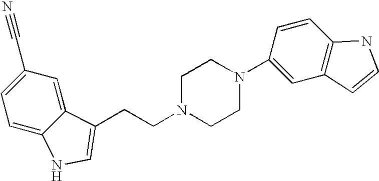 | 370 |

TLC (ethyl acetate / methanol 8:2): RF:0.5

| Melting point: | | 256.0-257.0° C. | | | |
|---|---|---|---|---|---|
| Elemental analysis: | | C | H | Cl | N |
| | Sought: | 68.05 | 5.96 | 8.73 | 17.25 |
| | Found: | 66.9 | 6.0 | 9.5 | 16.9 |

| | |
|---|---|
| 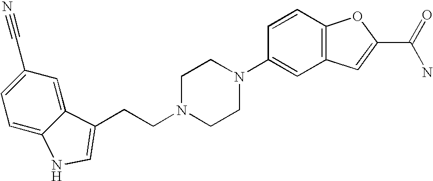 | 414 |

| | | | | | |
|---|---|---|---|---|---|
| Elemental analysis: | | C | H | Cl | N |
| | Sought: | 64.07 | 5.38 | 7.88 | 15.57 |
| | Found: | 62.7 | 5.5 | 8.0 | 14.8 |

| | |
|---|---|
| 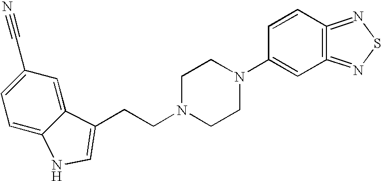 | 389 |

TLC (ethyl acetate / methanol 8:2): RF:0.5

| Melting point: | | 279.0-281.0° C. | | | | |
|---|---|---|---|---|---|---|
| Elemental analysis: | | C | H | Cl | N | S |
| | Sought: | 56.93 | 5.24 | 8.00 | 18.97 | 7.24 |
| | Found: | 56.7 | 5.4 | 7.7 | 19.0 | 7.6 |

(calculated on hydrochloride hydrate)

| | |
|---|---|
| 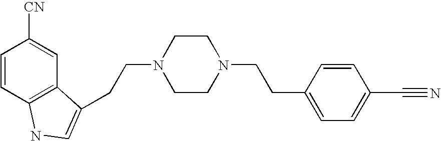 | 384 |

TLC (ethyl acetate / methanol 8:2): RF:0.3

| Melting point: | | 293.0-294.0° C. | | | |
|---|---|---|---|---|---|
| Elemental analysis: | | C | H | Cl | N |
| | Sought: | 63.15 | 5.97 | 15.53 | 15.35 |
| | Found: | 63.1 | 6.2 | 15.0 | 15.6 |

(calculated on dihydrochioride)

| | |
|---|---|
| 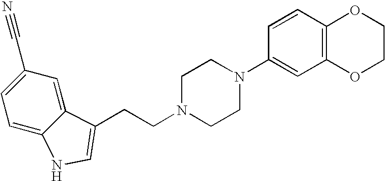 | 389 |
| 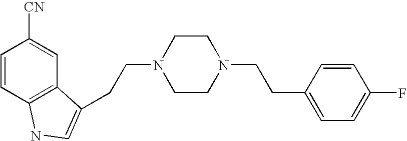 | 377 |

TLC (ethyl acetate / methanol 8:2): RF:0.4

| Melting point: | | 268.0-269.0° C. | | | | |
|---|---|---|---|---|---|---|
| Elemental analysis: | | C | H | Cl | F | N |
| | Sought: | 59.09 | 6.27 | 15.17 | 4.06 | 11.99 |
| | Found: | 58.7 | 6.5 | 13.2 | 4.5 | 11.9 |

(calculated on dihydrochioride)

| | |
|---|---|
| 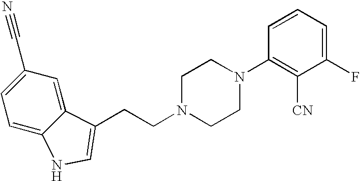 | 374 |

TLC (ethyl acetate / methanol 8:2): RF:0.4

| Melting point: | | 197.0-199.0° C. | | | | |
|---|---|---|---|---|---|---|
| Elemental analysis: | | C | H | Cl | F | N |
| | Sought: | 64.47 | 5.16 | 8.65 | 4.63 | 17.09 |
| | Found: | 62.1 | 5.5 | 8.7 | 4.5 | 16.5 |

| | |
|---|---|
| 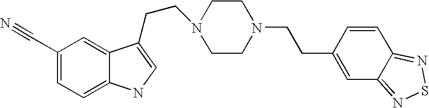 | 418 |

TLC (ethyl acetate / methanol 8:2): RF:0.3

| Melting point: | | 282.0-283.0° C. | | | | |
|---|---|---|---|---|---|---|
| Elemental analysis: | | C | H | Cl | N | S |
| | Sought: | 54.43 | 5.57 | 13.97 | 16.56 | 6.32 |
| | Found: | 54.5 | 5.6 | 13.0 | 16.4 | 6.9 |

(calculated on dihydrochloride hydrate)

| | |
|---|---|
| 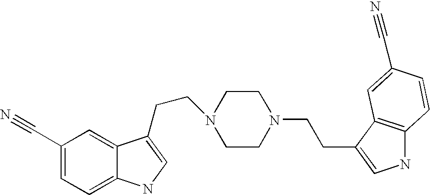 | 424 |

TLC (ethyl acetate / methanol 8:2): RF:0.2

| Melting point: | | 299.0-300.0° C. | | | |
|---|---|---|---|---|---|
| Elemental analysis: | | C | H | Cl | N |
| | Sought: | 60.81 | 5.98 | 13.81 | 16.37 |
| | Found: | 61.2 | 6.0 | 12.9 | 15.5 |

(calculated on dihydrochioride hydrate)

| | |
|---|---|
| 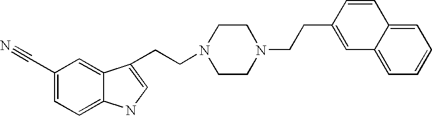 | 410 |

TLC (ethyl acetate / methanol 8:2): RF:0.3

| Melting point: | | 279.0-279.5° C. | | | |
|---|---|---|---|---|---|
| Elemental analysis: | | C | H | Cl | N |
| | Sought: | 64.92 | 6.47 | 14.19 | 11.22 |
| | Found: | 64.9 | 6.1 | 13.7 | 11.4 |

(calculated on dihydrochioride hydrate)

| | |
|---|---|
| 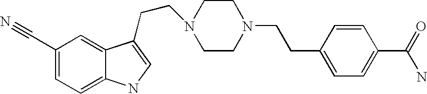 | 403 |

TLC (ethyl acetate / methanol 8:2): RF:0.1

| Melting point: | | 299.0-300.0° C. | | | |
|---|---|---|---|---|---|
| Elemental analysis: | | C | H | Cl | N |
| | Sought: | 60.76 | 6.16 | 14.95 | 14.76 |
| | Found: | 60.4 | 6.3 | 14.1 | 14.7 |

(calculated on dihydrochioride)

| | |
|---|---|
| 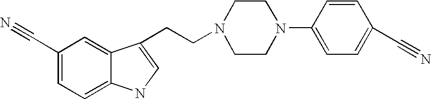 | 386 |

TLC (ethyl acetate / methanol 8:2): RF:0.4

| Melting point: | | 268.0-267.0° C. | | | |
|---|---|---|---|---|---|
| Elemental analysis: | | C | H | Cl | N |
| Sought: | 65.47 | 5.73 | 8.40 | 16.60 | |
| Found: | 63.1 | 5.8 | 7.8 | 15.6 | |

| | |
|---|---|
| 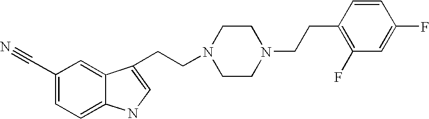 | 395 |

TLC (ethyl acetate / methanol 8:2): RF:0.4

| Melting point: | | 280.0-281.5° C. | | | | |
|---|---|---|---|---|---|---|
| Elemental analysis: | | C | H | Cl | F | N |
| | Sought: | 59.10 | 5.62 | 15.17 | 8.13 | 11.99 |
| | Found: | 58.5 | 5.6 | 15.5 | 11.8 | |

(calculated on dihydrochloride)

| | |
|---|---|
| 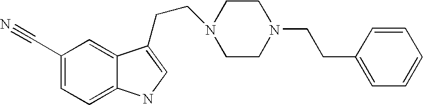 | 359 |

TLC (ethyl acetate / methanol 8:2): RF:0.3

| Melting point: | | 125.5-136.5° C. | | | |
|---|---|---|---|---|---|
| Elemental analysis: | | C | H | Cl | N |
| | Sought: | 59.89 | 6.91 | 13.17 | 11.99 |
| | Found: | 61.5 | 6.9 | 11.7 | 12.4 |

(calculated on dihydrochloride hydrate)

| | |
|---|---|
| 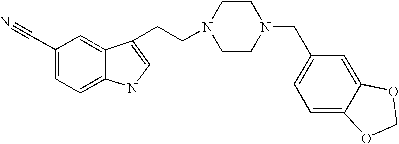 | 389 |

TLC (ethyl acetate / methanol 8:2): RF:0.4

| Melting point: | | 269.5-270.5° C. | | | |
|---|---|---|---|---|---|
| Elemental analysis: | | C | H | Cl | N |
| | Sought: | 59.86 | 5.69 | 15.37 | 12.14 |
| | Found: | 59.8 | 5.7 | 14.8 | 12.1 |

(calculated on dihydrochloride)

| | |
|---|---|
| 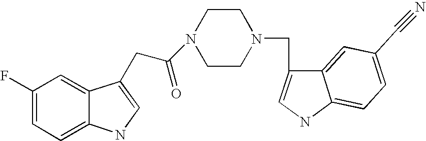 | 430 |

TLC (ethyl acetate / methanol 8:2): RF:0.3

| Melting point: | | 258.5-259.5° C. | | | | |
|---|---|---|---|---|---|---|
| Elemental analysis: | | C | H | Cl | F | N |
| | Sought: | 64.44 | 5.41 | 7.61 | 4.08 | 15.03 |
| | Found: | 63.8 | 5.6 | 7.9 | 3.8 | 14.6 |

| | |
|---|---|
| 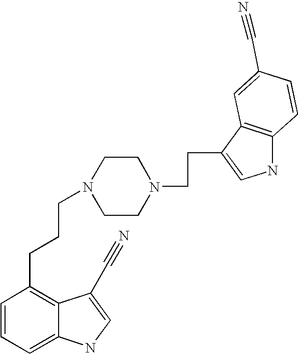 | 437 |

TLC (ethyl acetate / methanol 8:2): RF:0.2

| Melting point: | | 179.5-180.5° C. | | | |
|---|---|---|---|---|---|
| Elemental analysis: | | C | H | Cl | N |
| | Sought: | 57.54 | 6.45 | 12.58 | 14.92 |
| | Found: | 58.1 | 6.5 | 11.5 | 14.8 |

(calculated on dihydrochloride trihydrate)

| | |
|---|---|
| 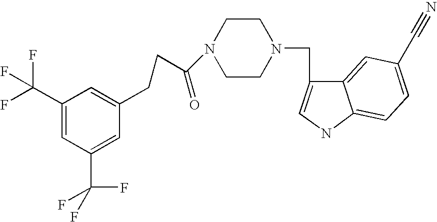 | 523 |

TLC (ethyl acetate / methanol 8:2): RF:0.4

| Melting point: | | 227.5-228.0° C. | | | | |
|---|---|---|---|---|---|---|
| Elemental analysis: | | C | H | Cl | F | N |
| | Sought: | 54.12 | 4.73 | 6.14 | 19.76 | 9.71 |
| | Found: | 54.5 | 5.1 | 6.0 | 15.9 | 10.1 |

(calculated on hydrochloride hydrate)

### EXAMPLE 6

### Receptor Binding Studies

As illustrative of two compounds of the formula I, receptor binding constants determined by the test systems described at the outset are indicated below:

a) 3-{2-[4-(2,3-Dihydrobenzo-1,4-dioxin-5-yl)piperazin-1-
yl]ethyl}-1H-indole-5-carbonitrile

| | |
|---|---|
| SSRI | 11 nmol/l |
| 5-HT_{1A} | 17 nmol/l |
| 5-HT_{2A} | 11 nmol/l |

b) 3-[2-(4-Benzo-1,2,5-thiadiazol-4-yl-piperazin-1-yl)ethyl]-
1H-indole-5-carbonitrile

| | |
|---|---|
| SSRI | 4.3 nmol/l |
| 5-HT_{1A} | 110 nmol/l |
| 5-HT_{2A} | 7.3 nmol/l |

The following examples relate to pharmaceutical compositions:

### EXAMPLE A

### Injection Vials

A solution of 100 g of an active ingredient of the formula I and 5 g of disodium hydrogenphosphate in 3 l of bidistilled water is adjusted to pH 6.5 using 2N hydrochloric acid, sterile filtered, transferred into injection vials, lyophilised and sealed under sterile conditions. Each injection vial contains 5 mg of active ingredient.

### EXAMPLE B

### Suppositories

A mixture of 20 g of an active ingredient of the formula I is melted with 100 g of soya lecithin and 1400 g of cocoa butter, poured into moulds and allowed to cool. Each suppository contains 20 mg of active ingredient.

### EXAMPLE C

### Solution

A solution is prepared from 1 g of an active ingredient of the formula I, 9.38 g of NaH₂PO₄×2H₂O, 28.48 g of NaH₂PO₄×12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE D

### Ointment

500 mg of an active ingredient of the formula I are mixed with 99.5 g of Vaseline under aseptic conditions.

### EXAMPLE E

### Tablets

A mixture of 1 kg of active ingredient of the formula I, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### EXAMPLE F

### Coated Tablets

Tablets are pressed analogously to Example E and subsequently coated in a conventional manner with a coating of sucrose, potato starch, talc, tragacanth and dye.

### EXAMPLE G

### Capsules

2 kg of active ingredient of the formula I are introduced into hard gelatine capsules in a conventional manner in such a way that each capsule contains 20 mg of the active ingredient.

### EXAMPLE H

### Ampoules

A solution of 1 kg of active ingredient of the formula I in 60 l of bidistilled water is transferred into ampoules, lyophilised under aseptic conditions and sealed under sterile conditions. Each ampoule contains 10 mg of active ingredient.

1. Compounds of the formula I 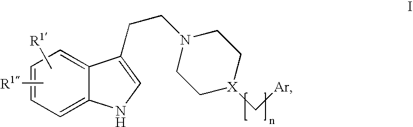
in which
R¹′, R¹″ each, independently of one another, denote H, CN, Hal, A, OA, OH, COR², CH₂R²,
R² denotes OH, OA, NH₂, NHA or NA₂,
R³ denotes H or A,
X denotes N or CH
A denotes unbranched or branched alkyl having 1-10 C atoms, in which one or two CH2 groups may be replaced by 0 or S atoms and/or by —CH═CH— groups and/or also 1-7H atoms may be replaced by F,
Ar denotes unsaturated, partially or fully saturated, mono- or polycyclic homo- or heterocyclic system containing the hetero atoms 0, N, S, which is unsubstituted or mono- or polysubstituted by Hal, A, OR³, N(R³)₂, N0₂, CN, COOR³, CON(R³)₂, NR³COA, NR³CON(R³)₂, NR³SO₂A, COR³, SO₂N(R³)₂, S0₂A,
Hal denotes F, Cl, Br or I and
n denotes 0, 1, 2, 3, 4
and pharmaceutically usable derivatives, solvates and stereoisomers thereof, including mixtures thereof in all ratios.

2. Compounds of the sub-formula Ia of the formula I according to claim 1, in which
R¹′ denotes cyano,
R¹″ denotes hydrogen,
X denotes N and
n denotes 0, 1 or 2
and solvates, stereoisomers and pharmaceutically usable derivatives thereof, including mixtures thereof in all ratios.

3. Compounds of the sub-formula Ib of the formula I according to claim 1, in which
R¹′ denotes cyano,
R¹″ denotes hydrogen,
X denotes N
n denotes 0, 1 or 2 and
Ar denotes phenyl which is unsubstituted or substituted in accordance with claim 1
and solvates, stereoisomers and pharmaceutically usable derivatives thereof, including mixtures thereof in all ratios.

4. Compounds of the sub-formula Ic of the formula I according to claim 1, in which
R¹′ denotes cyano,
R¹″ denotes hydrogen,
X denotes N
n denotes 0, 1 or 2 and
Ar denotes naphthyl which is unsubstituted or substituted as indicated in Claim 1
and solvates, stereoisomers and pharmaceutically usable derivatives thereof, including mixtures thereof in all ratios.

5. Compounds of the sub-formula Id of the formula I according to claim 1, in which
R¹′ denotes cyano,
R¹″ denotes hydrogen,
X denotes N
n denotes 0, 1 or 2 and
Ar denotes indolyl, benzofuryl or benzodioxolyl, each of which is unsubstituted or substituted as indicated in claim 1
and solvates, stereoisomers and pharmaceutically usable derivatives thereof, including mixtures thereof in all ratios.

6. Compounds of the sub-formula Ie of the formula I according to claim 1, in which
R¹′ denotes cyano,
R¹″ denotes hydrogen,
X denotes N
n denotes 0, 1 or 2 and
Ar denotes benzodioxinyl which is unsubstituted or substituted as indicated in claim 1
and solvates, stereoisomers and pharmaceutically usable derivatives thereof, including mixtures thereof in all ratios.

7. Compounds of the sub-formula If of the formula I according to claim 1, in which
R¹′ denotes cyano,
R¹″ denotes hydrogen,
X denotes N
n denotes 0, 1 or 2 and
Ar denotes benzothiadiazolyl which is unsubstituted or substituted as indicated in claim 1
and solvates, stereoisomers and pharmaceutically usable derivatives thereof, including mixtures thereof in all ratios.

8. Compounds of the formula I according to claim 1 selected from a group consisting of
(a) 3-{2-[4-(2,3-dihydrobenzo-1,4-dioxin-5-yl)piperazin-1-yl]ethyl}-1H-indole-5-carbonitrile,
(b) 3-[2-(4-benzo-1,2,5-thiadiazol-4-ylpiperazin-1-yl)ethyl]-1H-indole-5-carbonitrile.
and solvates, stereoisomers and pharmaceutically usable derivatives thereof, including mixtures thereof in all ratios.

9. Process for the preparation of compounds of the formula I according to claim 1 and pharmaceutically usable derivatives, solvates and stereoisomers thereof, characterised in that a formylindole starting material of the formula III 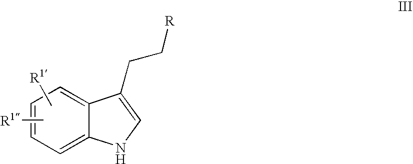
in which R is a leaving group which is suitable for nucleophilic substitutions, and R¹′ and R¹″ have a meaning indicated in claim 1, is reacted with a cycloamine compound of the formula II 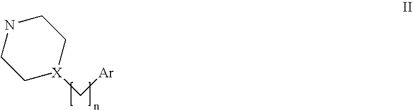
in which X, Ar, and n have the meaning indicated in claim.

10. Compounds of the formula I and pharmaceutically usable derivatives, solvates and stereoisomers thereof according to claim 1 as serotonin reuptake inhibitors and effectors of the serotonergic receptors 5-HT₁A and 5-HT₂A.

11. Compounds of the formula I and/or pharmaceutically usable derivatives, solvates and stereoisomers thereof, including mixtures thereof in all ratios according to claim 1 as medicaments.

12. Medicaments comprising at least one compound of the formula I and/or pharmaceutically usable derivatives, solvates and stereoisomers thereof, including mixtures thereof in all ratios according to claim 1, and optionally excipients and/or adjuvants.

13. Medicaments comprising at least one compound of the formula I and/or pharmaceutically usable derivatives, solvates and stereoisomers thereof, including mixtures thereof in all ratios according to claim 1, and at least one further medicament active ingredient.

14. Use of compounds according to claim 1 and/or pharmaceutically usable derivatives, solvates and stereoisomers thereof, including mixtures thereof in all ratios, for the preparation of a medicament for the prophylaxis or treatment of diseases in which inhibition of serotonin reuptake and/or binding of one or more active ingredients present in the said medicament to the serotonergic receptors 5-HT_{1A} and/or 5-HT_{2A} results in an improvement in the clinical picture.

15. Use of compounds according to claim 1 and/or pharmaceutically usable derivatives, solvates and stereoisomers thereof, including mixtures thereof in all ratios, for the preparation of a medicament for the prophylaxis or treatment of depression, dyskinesia, Parkinson's disease, dementia, strokes, schizophrenia, Alzheimer's disease, Lewy bodies dementia, Huntington's disease, Tourette's syndrome, anxiety, learning and memory impairment, sleeping disorders, pain and neurodegenerative diseases.

16. Pharmaceutical composition, characterised by a content of at least one compound of the formula I and/or pharmaceutically usable derivatives, solvates and stereoisomers thereof, including mixtures thereof in all ratios according to claim 1.

17. Process for the preparation of pharmaceutical compositions, characterised in that at least one compound of the formula I and/or pharmaceutically usable derivatives, solvates and stereoisomers thereof, including mixtures thereof in all ratios according to claim 1 is brought into a suitable dosage form together with at least one solid, liquid or semi-liquid excipient or adjuvant.

18. Set (kit) consisting of separate packs of
(a) an effective amount of a compound of the formula I according to claim 1 and/or pharmaceutically usable derivatives, solvates and stereoisomers thereof, including mixtures thereof in all ratios, and
(b) an effective amount of a further medicament active ingredient.

19. Use of compounds of the formula I and/or pharmaceutically usable derivatives, solvates and stereoisomers thereof, including mixtures thereof in all ratios according to claim 1, for the preparation of a medicament for the prophylaxis or treatment of depression, dyskinesia, Parkinson's disease, dementia, strokes, schizophrenia, Alzheimer's disease, Lewy bodies dementia, Huntington's disease, Tourette's syndrome, anxiety, learning and memory impairment, pain, sleeping disorders and neurodegenerative diseases, in combination with at least one further medicament active ingredient.

20. Intermediate compounds of the formula III 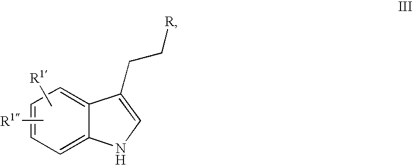
in which R is a leaving group which is suitable for nucleophilic substitutions, and R¹′, R¹″ have a meaning indicated in claim 1, and salts thereof.

21. Intermediate compounds of the formula III according to claim 20, consisting of 3-(2-chloroeth-1-yl)-1H-indole-5-carbonitrile and salts thereof.
